(19) United States
(12) Patent Application Publication
(10) Pub. No.: US 2024/0416086 A1
(43) Pub. Date: Dec. 19, 2024

(54) ARTERIAL HEMOSHIELD DEVICE FOR ARTERIAL BLOOD DRAW AND RELATED METHODS

(71) Applicant: Becton, Dickinson and Company, Franklin Lakes, NJ (US)

(72) Inventors: Yiping Ma, Layton, UT (US); Jonathan Karl Burkholz, Salt Lake City, UT (US); Christopher Anthony Basciano, Apex, NC (US); Sivaramakrishnan Balasubramanian, Cary, NC (US); Siddharth Nagarajan, Bangalore (IN)

(21) Appl. No.: 18/677,611

(22) Filed: May 29, 2024

Related U.S. Application Data
(60) Provisional application No. 63/521,028, filed on Jun. 14, 2023.

(51) Int. Cl. A61M 25/06; A61M 25/00; A61M 39/02; A61M 39/10

(52) U.S. Cl. CPC A61M 25/0606; A61M 2025/0031; A61M 2039/0258; A61M 2039/1083; A61M 2039/1088; A61M 2210/12

(57) ABSTRACT

An arterial catheter system may include a catheter assembly, which may include a catheter adapter. The arterial catheter system may include an arterial catheter extending from a distal end of the catheter adapter. The arterial catheter system may include a needle assembly, which may include a needle hub and an introducer needle. The arterial catheter system may include an arterial hemoshield device coupled to the catheter assembly. The arterial catheter system may include a fluid pathway within the arterial catheter, the catheter adapter, and the arterial hemoshield device. A first fluidic resistance within a portion of the fluid pathway within the arterial hemoshield device may be greater than a second fluidic resistance within the fluid pathway distal to the portion of the fluid pathway.

### RELATED APPLICATIONS

This application claims the benefit of U.S. Provisional Patent Application No. 63/521,028, which was filed on Jun. 14, 2023, which is incorporated herein in its entirety.

### BACKGROUND

A catheter is commonly used to infuse fluids into vasculature of a patient. For example, the catheter may be used for infusing normal saline solution, various medicaments, or total parenteral nutrition. The catheter may also be used for withdrawing blood from the patient.

The catheter may include an over-the-needle intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of a catheter assembly including the catheter. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection container may be used. The blood collection container may include a syringe or a test tube with a rubber stopper at one end. In some instances, the blood collection container has had all or a portion of air removed from the test tube so pressure within the blood collection container is lower than ambient pressure. Such a blood collection container is often referred to as an internal vacuum or a vacuum tube. A commonly used blood collection container is a VACUTAINER® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills, until the pressure in the blood collection container equalizes with the pressure in the vein, and the flow of blood stops.

Unfortunately, as blood is drawn into the blood collection container, red blood cells are in a high shear stress state and susceptible to hemolysis due to a high initial pressure differential between the vein and the blood collection container. Hemolysis may result in rejection and discard of a blood sample. The high initial pressure differential can also result in catheter tip collapse, vein collapse, or other complications that prevent or restrict blood from filling the blood collection container. As the blood collection container fills, a pressure differential between the vein and the blood collection container decreases, and filling of the blood collection tube with blood slows significantly. The risk for hemolysis is much higher in the case of arterial blood draw due to higher pressure in the artery in comparison to venous blood draw.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure generally relates to an arterial catheter system configured for blood draw or collection, as well as related devices and methods. In some embodiments, the arterial catheter system may include a catheter assembly, which may include a catheter adapter and an arterial catheter. In some embodiments, the catheter adapter may include a distal end and a proximal end. In some embodiments, the arterial catheter may extend from the distal end of the catheter adapter.

In some embodiments, the arterial catheter system may include a needle assembly, which may include a needle hub and an introducer needle extending from the needle hub. In some embodiments, the arterial catheter system may include an arterial hemoshield device coupled to the catheter assembly. In some embodiments, the arterial catheter system may include a fluid pathway within the arterial catheter, the catheter adapter, and the arterial hemoshield device.

In some embodiments, a first fluidic resistance within a portion of the fluid pathway within the arterial hemoshield device may be greater than a second fluidic resistance within the fluid pathway distal to the portion of the fluid pathway. In some embodiments, the first fluidic resistance within the portion of the fluid pathway may facilitate a decreased flow rate of arterial blood within the portion of the fluid pathway such that the maximum shear stress is reduced and there is a decreased risk of hemolysis of the arterial blood that will be collected.

In some embodiments, the arterial hemoshield device may include a distal end, which may include a luer adapter. In some embodiments, the arterial hemoshield device may include a proximal end, which may include a blood collection device. In some embodiments, the arterial hemoshield device may include an extension tube extending between the distal end and the proximal end. In some embodiments, the portion of the fluid pathway may be disposed within the extension tube.

In some embodiments, the extension tube may include a distal end and a proximal end. In some embodiments, the distal end of the extension tube may be integrated with the luer adapter. In some embodiments, the proximal end of the extension tube may be integrated with the blood collection device. In some embodiments, the luer adapter may be a first luer adapter. In some embodiments, the blood collection device may include a second luer adapter. In some embodiments, the arterial catheter system may include a third luer adapter coupled to the second luer adapter. In some embodiments, the extension tube may include a distal end and a proximal end. In some embodiments, the distal end of the extension tube may be integrated with the first luer adapter. In some embodiments, the proximal end of the extension tube may be integrated with the third luer adapter. In some embodiments, a geometric factor G_{f} of the portion of the fluid pathway may be different than a geometric factor G_{f} of another portion of the fluid pathway. In some embodiments, the extension tube may have no more than one lumen extending therethrough.

In some embodiments, the arterial hemoshield device may include a compact connector, which may include a spiral tube. In some embodiments, the portion of the fluid pathway may be disposed within the spiral tube. In some embodiments, the catheter adapter may include a side port disposed between the distal end of the catheter adapter and the proximal end of the catheter adapter. In some embodiments, the catheter assembly may include another extension tube extending from the side port. In some embodiments, a distal end of the another extension tube may be integrated with the side port. In some embodiments, a proximal end of the other extension tube may be integrated with a Y-adapter. In some embodiments, the compact connector may be coupled to the Y-adapter. In some embodiments, a geometric factor G_{f} of the portion of the fluid pathway is different than a geometric factor G_{f} of another portion of the fluid pathway. In some embodiments, the spiral tube may have no more than one lumen extending therethrough.

In some embodiments, the arterial hemoshield device may include a female luer adapter coupled to catheter assembly. In some embodiments, the arterial hemoshield device may include a blood collection device, which may include a distal end. In some embodiments, the distal end of the blood collection device may include a male luer adapter coupled to the female luer adapter. In some embodiments, the male luer adapter may include a distal opening. In some embodiments, the arterial hemoshield device may include a cannula in fluid communication with the male luer adapter. In some embodiments, the cannula may include a distal end and a sharp proximal tip. In some embodiments, an elongated neck may be disposed between the male luer adapter and the sharp proximal tip. In some embodiments, the portion of the fluid pathway extends from the distal opening through the sharp proximal tip. In some embodiments, a geometric factor G_{f} of the portion of the fluid pathway may be different than a geometric factor G_{f} of another portion of the fluid pathway.

In some embodiments, a method of blood collection may include inserting the arterial catheter of the arterial catheter system into an artery of a patient. In some embodiments, the arterial catheter system may include the catheter assembly. In some embodiments, the catheter assembly may include the catheter adapter, which may include the distal end and the proximal end. In some embodiments, the catheter assembly may include the arterial catheter extending from the distal end of the catheter adapter.

In some embodiments, the arterial catheter system may include the needle assembly, which may include the needle hub and the introducer needle extending from the needle hub. In some embodiments, the arterial catheter system may include the arterial hemoshield device coupled to the catheter assembly. In some embodiments, the arterial catheter system may include the fluid pathway within the arterial catheter, catheter adapter, and the arterial hemoshield device. In some embodiments, the first fluidic resistance within the portion of the fluid pathway within the arterial hemoshield device may be greater than the second fluidic resistance within the fluid pathway distal to the portion of the fluid pathway.

In some embodiments, the method of blood collection may include collecting arterial blood in a particular blood collection device, which may be coupled to the catheter assembly, whereby the arterial blood flows through the portion of the fluid pathway and into the particular blood collection device.

In some embodiments, a method of manufacturing the arterial catheter system may include coupling the catheter assembly to the needle assembly. In some embodiments, the method of manufacturing may include coupling an arterial hemoshield device to the catheter assembly such that the arterial hemoshield device is in fluid communication with the catheter assembly and a portion of the fluid pathway is within the arterial catheter, the catheter hub, and the arterial hemoshield device.

In some embodiments, the method of manufacturing may include selecting a length L of a portion of the fluid pathway within the hemoshield device and an inner diameter D of the portion of the fluid pathway within the hemoshield device such that a first fluidic resistance within the portion of the fluid pathway is greater than a second fluidic resistance within the fluid pathway distal to the portion of the fluid pathway.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example arterial hemoshield device, according to some embodiments;

FIG. 1B is a cross-sectional view of the arterial hemoshield device of FIG. 1A, according to some embodiments;

FIG. 1C is an upper perspective view of the arterial hemoshield device of FIG. 1A coupled to an example arterial catheter assembly, according to some embodiments;

FIG. 2A is an upper perspective view of another example arterial hemoshield device, according to some embodiments;

FIG. 2B is a cross-sectional view of the arterial hemoshield device of FIG. 2B, according to some embodiments;

FIG. 2C is an upper perspective view of the arterial hemoshield device of FIG. 2A coupled to the arterial catheter assembly of FIG. 1C, according to some embodiments;

FIG. 3A is an upper perspective view of an example arterial catheter system, according to some embodiments; and

FIG. 3B is an upper perspective view of an example portion of an arterial catheter system, according to some embodiments;

FIG. 4A is an upper perspective view of an example arterial catheter system, illustrating an example compact connector, according to some embodiments;

FIG. 4B is a cross-sectional view of a portion of the arterial catheter system of FIG. 4A, according to some embodiments;

FIG. 5A is an upper perspective view of an example arterial hemoshield device, according to some embodiments;

FIG. 5B is a cross-sectional view of the arterial hemoshield device of FIG. 5A, according to some embodiments;

FIG. 5C is an upper perspective view of a portion of the arterial hemoshield device of FIG. 5A, according to some embodiments;

FIG. 5D is a cross-sectional view of the portion coupled to a remaining portion of the arterial hemoshield device of FIG. 5A, according to some embodiments;

FIG. 6A is an upper perspective view of an example arterial catheter system, according to some embodiments;

FIG. 6B is a cross-sectional view of the arterial catheter system of FIG. 3A, illustrating an example needle assembly removed, according to some embodiments.

### DESCRIPTION OF EMBODIMENTS

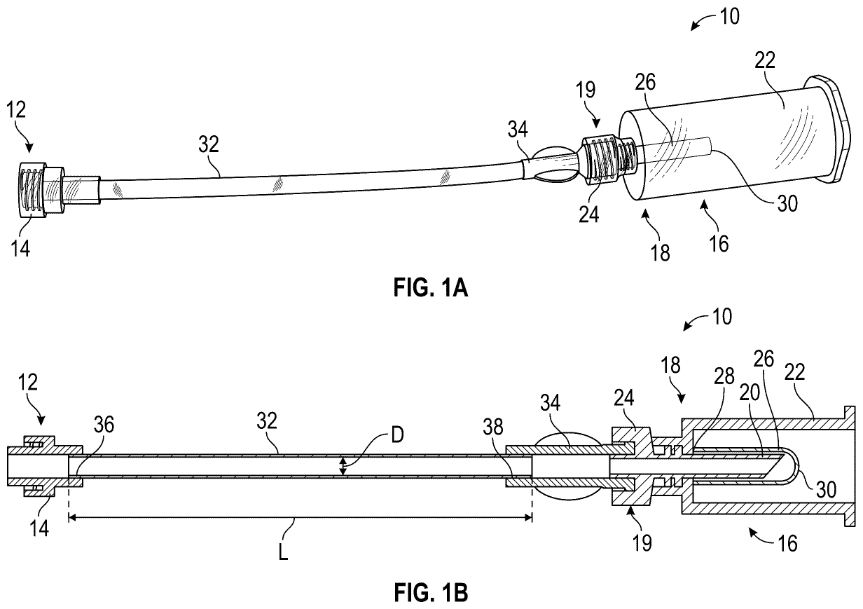

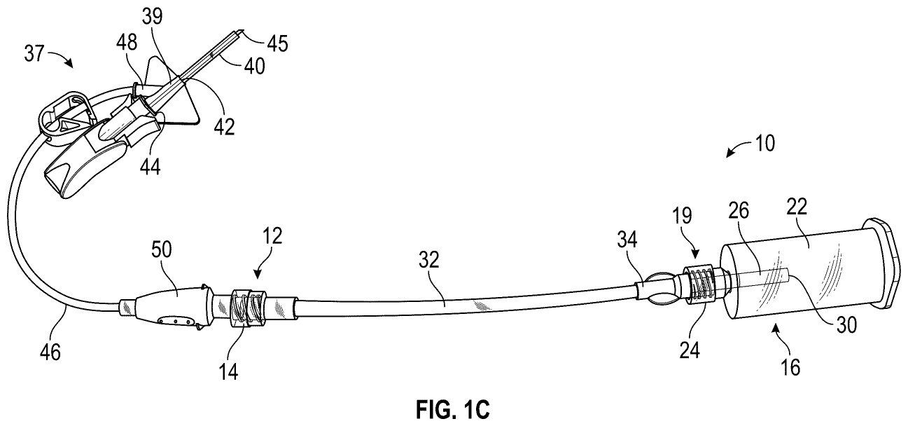

Referring now to FIGS. 1A-1C, in some embodiments, an arterial hemoshield device 10 may include a distal end 12, which may include a luer adapter 14 configured to couple to a catheter adapter or another suitable vascular access device. In some embodiments, the arterial hemoshield device 10 may be configured to reduce a maximum shear stress on arterial blood drawn from an artery into an arterial catheter system and thereby reduce a risk of hemolysis of the arterial blood, providing an improved blood sample. In some embodiments, the arterial hemoshield device 10 may include a proximal end 16, which may include a blood collection device 18. In some embodiments, the blood collection device 18 may include or correspond to a blood collection container. In some embodiments, the blood collection container may include a syringe, an evacuated blood collection tube (or vacuum tube), a small sample collection device, or any other container configured to collect blood from a patient via a pressure differential.

In some embodiments, the blood collection device 18 may include a needle assembly 19, which may include a needle 20 configured to receive a blood collection container. In these and other embodiments, the blood collection container may include the evacuated blood collection tube. In these embodiments, the blood collection container may have all or a portion of air removed so pressure within the blood collection container is lower than ambient pressure.

In some embodiments, the needle assembly 19 may include one or more threads, which may be configured to couple to a holder 22 of the blood collection device 18, which may be generally cylindrical and may be configured to hold the blood collection container. In some embodiments, the holder 22 may be integrally formed with the needle assembly 19 or coupled to the needle assembly 19 via bonding or another suitable method. In some embodiments, the holder 22 may surround the needle 20. In some embodiments, the needle assembly 19 and the holder 22 may include or correspond to a luer lock access device, such as, for example, the VACUTAINER® LUER-LOK™ ACCESS DEVICE available from Becton Dickinson & Company. In some embodiments, the holder 22 may include or correspond to the blood collection tube holder 127 described in U.S. patent application Ser. No. 17/075,420, filed Oct. 20, 2020, entitled “BLOOD COLLECTION SYSTEM WITH USER-ADJUSTED PRESSURE MANAGEMENT AND RELATED METHODS,” which is incorporated by reference in its entirety.

In some embodiments, the luer adapter 14 may be a first luer adapter. In some embodiments, the arterial hemoshield device 10 may include a second luer adapter 24. In some embodiments, the blood collection device 18 may include the second luer adapter 24. In some embodiments, the needle 20 may be integrated with the second luer adapter 24. In some embodiments, a proximal end of the needle 20 may be enveloped within an elastomeric sheath 26. In some embodiments, the elastomeric sheath 26 may include an open distal end 28 and a closed proximal end 30. In some embodiments, in response to the blood collection container pushing the elastomeric sheath 26 distally, the needle 20 may pierce the elastomeric sheath 26, and the needle 20 may insert into a cavity of the blood collection container.

In some embodiments, the arterial hemoshield device 10 may include an extension tube 32, which may extend between the distal end 12 of the arterial hemoshield device 10 and the proximal end 16 of the arterial hemoshield device 10. In some embodiments, the extension tube 32 may be rigid or semi-rigid, which may reduce a likelihood of kinking. In some embodiments, the extension tube 32 may be flexible such that it is configured to bend. In some embodiments, the extension tube 32 may be constructed of plastic. In some embodiments, the extension tube 32 may include no more than one lumen extending therethrough.

In some embodiments, the arterial hemoshield device 10 may include a third luer adapter 34, which may be coupled to the second luer adapter 24. In some embodiments, the extension tube 32 may include a distal end 36 and a proximal end 38. In some embodiments, the distal end 36 may be coupled to or integrated with the first luer adapter. In some embodiments, the proximal end of the extension tube 32 may be coupled to or integrated with the third luer adapter 34. In other embodiments, the proximal end of the extension tube 32 may be coupled to or integrated with the blood collection device 18 and the arterial catheter system may not include the third luer adapter 34.

In some embodiments, the arterial hemoshield device 10 may act as a flow resistor in a fluid pathway of an arterial catheter system, illustrated, for example, in FIG. 1C, or another vascular access system. In some embodiments, the arterial catheter system may include an arterial catheter assembly 37, which may include a catheter adapter 39 and an arterial catheter 40. In some embodiments, the arterial catheter 40 may be secured within the catheter adapter 39 and may extend distally from the catheter adapter 39. In some embodiments, the catheter adapter 39 may include a distal end 42, a proximal end 44, and a lumen extending through the distal end 42 and the proximal end 44. In some embodiments, an introducer needle 45 may extend from a needle shield through the arterial catheter 40.

In some embodiments, the arterial catheter assembly 37 may be integrated. In further detail, in some embodiments, another extension tube 46 may extend from a side port 48 the catheter adapter 39. In some embodiments, a proximal end of the other extension tube 46 may include a fourth luer adapter 50, which may be coupled to the first luer adapter. In some embodiments, the arterial catheter assembly 37 may be straight and/or the first luer adapter may be coupled to the proximal end 44 of the catheter adapter 39. In some embodiments, one or more of the first luer adapter, the second luer adapter 24, the third luer adapter 34, and the fourth luer adapter 50 may include a slip or thread or clip male luer adapter, a slip or thread female luer adapter, a needleless connector, a blunt cannula, or another suitable access device.

In some embodiments, the arterial catheter 40 may include an arterial catheter. In some embodiments, the arterial catheter 40 may be shorter and/or more rigid than an intravenous catheter, such as a peripheral intravenous catheter. In some embodiments, the arterial hemoshield device 10 may be coupled to the arterial catheter assembly 37 in any number of suitable ways. In some embodiments, the fluid pathway of the arterial catheter system may include one or more of the following: the arterial catheter 40, the catheter adapter 39, the other extension tube 46, the fourth luer adapter 50, the first luer adapter, the extension tube 32, the third luer adapter 34, the second luer adapter 24, and the blood collection device 18. In some embodiments, the arterial hemoshield device 10 may lower a flow rate of blood within the fluid pathway of the arterial catheter system, which may in turn lower a shear rate for hemolysis management. In some embodiments, the arterial catheter assembly 37 may be substituted with another type of vascular access device, such as, for example, a venipuncture device, an infusion disposable, a blood collection access device, or a blood collection container.

In some embodiments, a geometric factor G_{f} of a portion of the fluid pathway within the extension tube 32 may equal L/D⁴, wherein L is the length of the extension tube 32 and D is the inner diameter of the portion of the fluid pathway within the extension tube 32. In these embodiments, the portion of the fluid pathway may be cylindrical along an entirety of the length L, and the inner diameter D may be constant along the length L. The length L corresponds to an entire length of the extension tube 32, according to some embodiments. In some embodiments, the geometric factor G_{f} of the portion of the fluid pathway within the extension tube 32 may be defined such that a fluidic resistance

$${R}_{f} = \frac{128 μ}{π} {G}_{f} ,$$

where

$${G}_{f} = \frac{L}{{D}^{4}} .$$

In some embodiments, the geometric factor G_{f} of the portion of the fluid pathway may be selected to reduce a maximum shear stress on arterial blood drawn from an artery and thereby reduce a risk of hemolysis of the arterial blood.

A blood cell experiences shear stress as it flows in a fluid pathway. The maximum shear stress is along the wall of the fluid pathway, or wall shear stress. Wall shear stress on blood cells is considered a major source of mechanical damage to blood cells. For a cylindrical fluid path, the wall shear stress is typically expressed as:

$$τ = \frac{1}{2} \cdot \frac{Δ p}{L} \cdot ( kr )$$

in which ΔP is the pressure drop along a path with a length of L and an interior radius of r. k is shrinkage index.

To fill a certain volume of collection tube, V, with a flow rate of Q, the time needed can be simply assessed by:

$$t = \frac{v}{ℚ} = 8 μv \cdot \frac{1}{π {r}^{4}} / ( \frac{Δ p}{L} )$$

in which μ is the dynamic viscosity of the fluid. Hemolysis is typically associated with both the wall shear stress and the time a blood cell is exposed to wall shear stress. From literature, it has been widely considered that hemolysis index can be approached as a function of:

$$HI ( \% ) = A * {t}^{α} * {τ}^{β}$$

in which A, α, and β are coefficients.

In principle, the hemolysis index is related to pressure gradient and cross-sectional characteristic dimension:

$$HI ( \% ) ∝ {( \frac{Δ P}{i} )}^{β - α} \cdot {( \frac{1}{r} )}^{4 α - β}$$

Fluid flow in a particular extension tube with a cylindrical fluid pathway therethrough can be analyzed using Poiseuille's equation:

$$Q = \frac{π {D}^{4} Δ P}{128 μ L} = \frac{Δ P}{{R}_{f}}$$

where ΔP is a change in pressure gradient across the length of the particular extension tube, D and L are the inner diameter and length, respectively, of the cylindrical fluid pathway through the particular extension tube, μ is the viscosity of a fluid, and

$${R}_{f} = \frac{128 μ L}{π {D}^{4}}$$

is the fluidic resistance. The particular extension tube may include or correspond to the extension tube 32. Since μ is the viscosity of the fluid and not part of the extension tube geometry, the geometric factor G_{f} is defined such that R_{f} (the fluidic resistance) is

$${R}_{f} = \frac{128 μ}{π} {G}_{f} ,$$

where

$${G}_{f} = \frac{L}{{D}^{4}} .$$

In some embodiments, the extension tube 32 may have multiple sections with lengths (L1, L2, L3) and inner diameters of (D1, D2, D3), the geometric factor is then:

$${G}_{f} = \frac{L 1}{D {1}^{4}} + \frac{L 2}{D {2}^{4}} + \frac{L 3}{D {3}^{4}}$$

In some embodiments, the extension tube 32 may have an inner diameter that changes over the length of the extension tube, the geometric factor is then:

$${G}_{f} = {\int }_{0}^{L} \frac{d l}{{D ( l )}^{4}}$$

In some embodiments, the extension tube 32 may have a cross section that is not circular. In this case, the geometric factor can be determined by measuring the flow rate (Q) at given pressure (ΔP) with known viscosity (μ) fluid:

$${G}_{f} = \frac{π Δ P}{128 μ Q}$$

In some embodiments, the fluidic resistance may be a first fluidic resistance. In some embodiments, the first fluidic resistance of the portion of the fluid pathway within the extension tube may be greater than a second fluidic resistance within the fluid pathway distal to the portion of the fluid pathway. For example, the first fluidic resistance may be greater than a particular fluidic resistance within a lumen of the catheter adapter 39, where arterial blood may travel prior to reaching the extension tube 32 and the arterial hemoshield device 10 through which blood may be collected.

In some embodiments, the arterial catheter 40 may be a 20 G arterial catheter. In these embodiments, the length L and the inner diameter D may be selected such that the geometric factor G_{f} of the portion of the fluid pathway within the extension tube 32 is 3.41E+06 (1/in³) or higher. In some embodiments, the length L and the inner diameter D for a 20 G arterial catheter may be selected such that the geometric factor G_{f} of the portion of the fluid pathway within the extension tube 32 is 3.41E+06 (1/in³)+/−10%.

In some embodiments, the arterial catheter 20 may be a 18 G arterial catheter. In these embodiments, the length L and the inner diameter D may be selected such that the geometric factor G_{f} of the portion of the fluid pathway within the extension tube 32 is 2.88E+06 (1/in³) or higher. In some embodiments, the length L and the inner diameter D for a 18 G arterial catheter may be selected such that the geometric factor G_{f} of the portion of the fluid pathway within the extension tube 32 is 2.88E+06 (1/in³)+/−10%.

In some embodiments, the arterial catheter 20 may be a 22 G arterial catheter. In these embodiments, the length L and the inner diameter D may be selected such that the geometric factor G_{f} of the portion of the fluid pathway within the extension tube 32 is 1.05E+07 (1/in³) or higher. In some embodiments, the length L and the inner diameter D for a 24 G arterial catheter may be selected such that the geometric factor G_{f} of the portion of the fluid pathway within the extension tube 32 is 1.05E+07 (1/in³)+/−10%.

In some embodiments, the arterial catheter 20 may be a 24 G arterial catheter. In these embodiments, the length L and the inner diameter D may be selected such that the geometric factor G_{f} of the portion of the fluid pathway within the extension tube 32 is 3.20E+07 (1/in³) or higher. In some embodiments, the length L and the inner diameter D for a 24 G arterial catheter may be selected such that the geometric factor G_{f} of the portion of the fluid pathway within the extension tube 32 is 3.20E+07 (1/in³)+/−10%.

In some embodiments, the fluid pathway of the arterial catheter system, which may include one or more of the needle assembly 19, the extension tube 32, and the arterial catheter assembly 37 (which may include the other extension tube 46), may include an entirety of a blood collection pathway through which blood flows during blood collection. The system geometric factor G_{fs} for the fluid pathway of the arterial catheter system can be determined in similar fashion as described earlier.

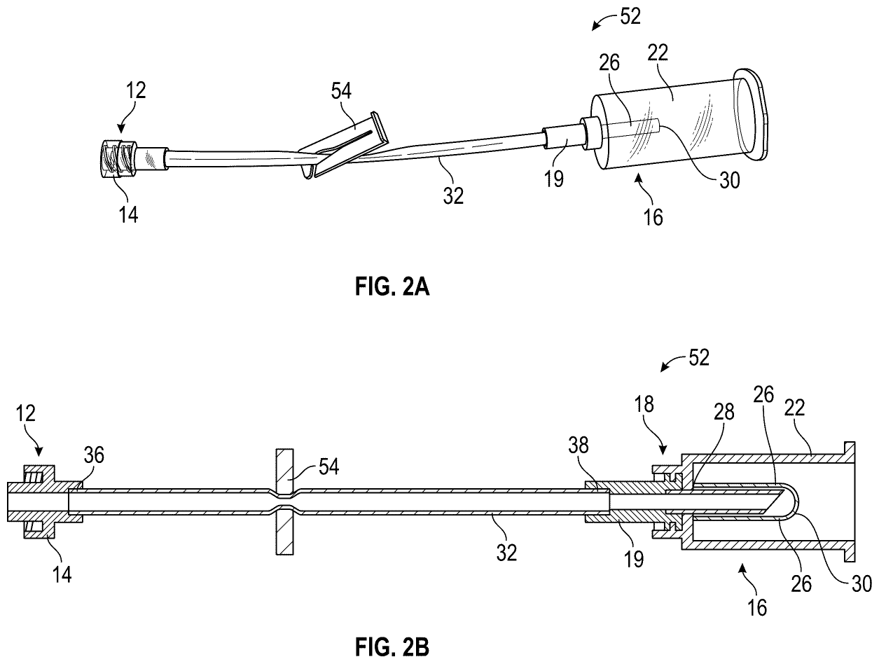

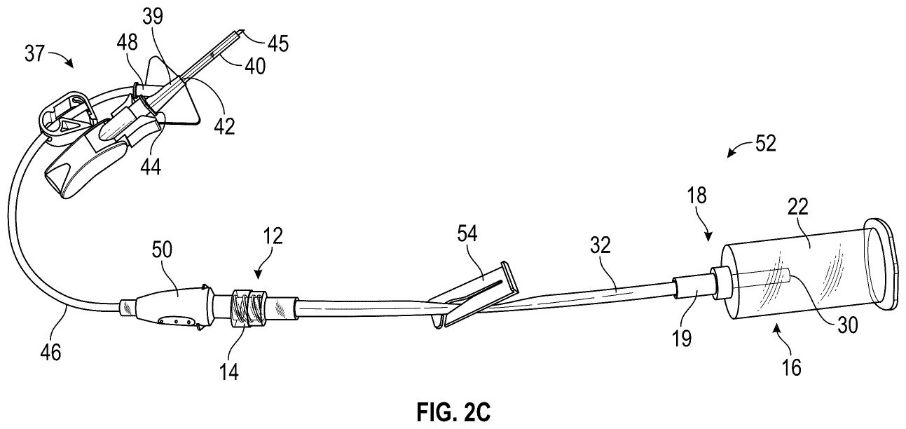

Referring now to FIGS. 2A-2C, an arterial hemoshield device 52 is illustrated, according to some embodiments. In some embodiments, the arterial hemoshield device 52 may be similar or identical to the arterial hemoshield device 10 of FIGS. 1A-1C in terms of one or more included features and/or operation. In some embodiments, the proximal end of the extension tube 32 may be integrated with the blood collection device 18. In some embodiments, the arterial hemoshield device 52 may include a clamp 54, which may be disposed on the extension tube 32. In some embodiments, the clamp 54 may be configured to move between a clamped position and an unclamped position or between a more clamped position and a less clamped position. In some embodiments, the clamp 54 may prevent or reduce fluid flow through the extension tube 32 in response to the clamp 54 being in the clamped position.

In some embodiments, the clinician may adjust flow resistance within the arterial catheter system by manually changing fluid characteristics of the arterial catheter system via the clamp 54. In some embodiments, in response to the clamp 54 being in the clamped position, flow resistance within the arterial catheter system may be increased and blood flow through the extension tube 32 may be reduced. In these embodiments, a risk of hemolysis may be reduced. In some embodiments, to decrease the flow resistance within the arterial catheter system after the blood collection container nears filling, the clinician may move the clamp to the unclamped position, which may allow faster blood collection when a risk of hemolysis is reduced.

In some embodiments, the clamp 54 may include a slide clamp, which may include a slot that becomes progressively narrower. In these and other embodiments, the extension tube 32 may be flexible and compliant. In some embodiments, the clinician may adjust an inner diameter of the extension tube 32 by adjusting a depth of the extension tube 32 within the slot of the slide clamp. The clinician may in turn adjust a flow resistance within the arterial hemoshield device 52. In some embodiments, the clamp 54 may include a roller clamp, a slide clamp, a pinch clamp, or another suitable type of clamp.

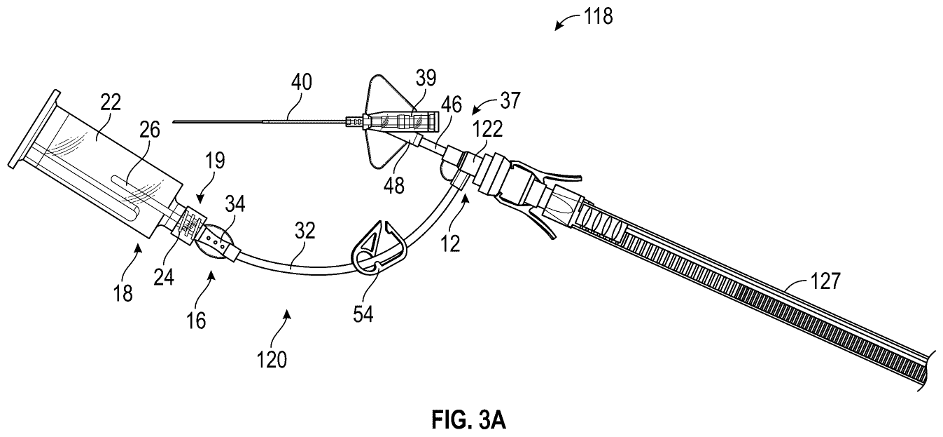

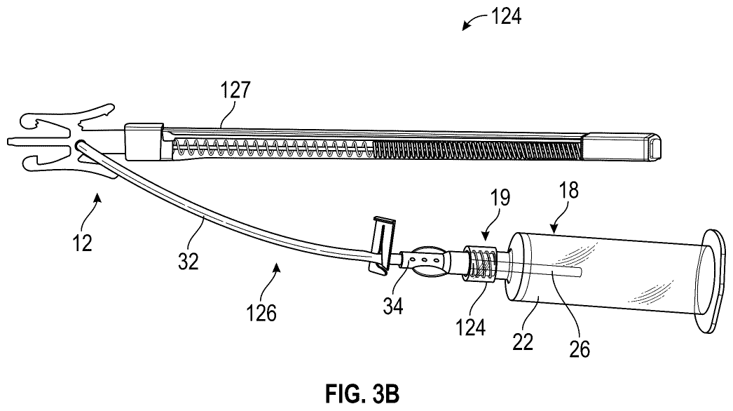

Referring now to FIG. 3A, an arterial catheter assembly 118 is illustrated, according to some embodiments. In some embodiments, the arterial catheter assembly 118 may include an arterial hemoshield device 120, which may be integrated with the arterial catheter assembly 37. In further detail, in some embodiments, the distal end 12 of the arterial hemoshield device 120 may be integrated with an adapter 122 of the arterial catheter assembly 37, as illustrated, for example, in FIG. 3A, or integrated with the catheter adapter 39 itself. In these and other embodiments, the arterial hemoshield device 120 may not be removable from the arterial catheter assembly 118. In some embodiments, the distal end of the extension tube 32 may be integrated with the adapter 122 or the catheter adapter 39. In some embodiments, the adapter 122 may include a Y-adapter, a T-adapter, or another suitable adapter. In some embodiments, the other extension tube 46 may be shorter than the extension tube 52 such that the adapter 122 provides a port for near patient blood collection or sampling. In some embodiments, the arterial hemoshield device 120 may be similar or identical to one or more of the following in terms of one or more included features and/or operation: the arterial hemoshield device 10 of FIGS. 1A-1C and the arterial hemoshield device 52 of FIGS. 2A-2C.

Referring now to FIG. 3B, a portion 124 of an arterial catheter assembly is illustrated, according to some embodiments. In some embodiments, the portion 124 of the arterial catheter assembly may include an arterial hemoshield device 126, which may be coupled to or integrated with an instrument delivery device 127, which may deliver a probe, a catheter, or a guidewire through a particular arterial catheter assembly (as illustrated, for example, in FIG. 3A). In some embodiments, the arterial hemoshield device 126 may be similar or identical to one or more of the following in terms of one or more included features and/or operation: the arterial hemoshield device 10 of FIGS. 1A-1C and the arterial hemoshield device 52 of FIGS. 2A-2C.

In some embodiments, the instrument delivery device 127 may include any suitable instrument delivery device. In some embodiments, the instrument delivery device 127 may be further described in U.S. Pat. No. 11,969,247, granted Apr. 30, 2024, entitled “EXTENSION HOUSING A PROBE OR INTRAVENOUS CATHETER,” U.S. patent application Ser. No. 16/388,650, filed Apr. 18, 2019, entitled “INSTRUMENT DELIVERY DEVICE HAVING A ROTARY ELEMENT,” U.S. Pat. No. 11,173,277, granted Nov. 16, 2021, entitled “MULTI-DIAMETER CATHETER AND RELATED DEVICES AND METHODS,” U.S. Pat. No. 11,406,795, filed Aug. 9, 2022, entitled “DELIVERY DEVICE FOR A VASCULAR ACCESS INSTRUMENT,” U.S. Pat. No. 11,337,628, granted May 24, 2022, entitled “SYRINGE-BASED DELIVERY DEVICE FOR A VASCULAR ACCESS INSTRUMENT,” U.S. Pat. No. 11,547,832, granted Jan. 10, 2023, entitled “CATHETER DELIVERY DEVICE AND RELATED SYSTEMS AND METHODS,” and U.S. Pat. No. 11,504,503, granted Nov. 22, 2022, entitled “VASCULAR ACCESS INSTRUMENT HAVING A FLUID PERMEABLE STRUCTURE AND RELATED DEVICES AND METHODS,” which are incorporated by reference in their entirety.

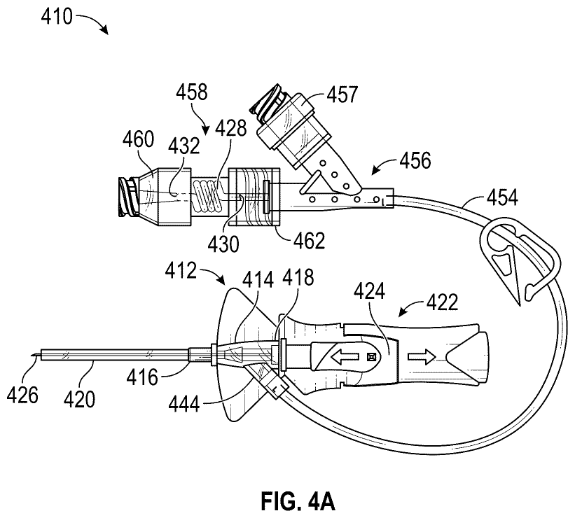

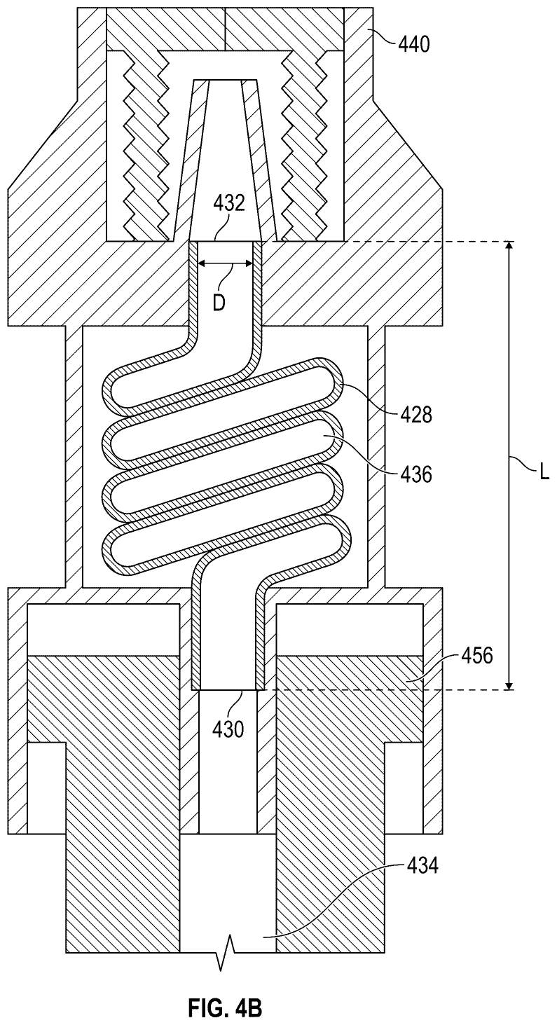

Referring now to FIGS. 4A-4B, an arterial catheter system 410 is illustrated, according to some embodiments. In some embodiments, the arterial catheter system 410 may be configured to reduce a maximum shear stress on arterial blood drawn from an artery into the arterial catheter system 410 and thereby reduce a risk of hemolysis of the arterial blood, providing an improved blood sample. In some embodiments, the arterial catheter system 410 may be similar or identical to the arterial catheter system of one or more of FIGS. 1C, 3A, and 3B in terms of one or more included features and/or operation.

In some embodiments, the arterial catheter system 410 may include an arterial catheter assembly 412, which may include a catheter adapter 414. In some embodiments, the catheter adapter 414 may include a distal end 416 and a proximal end 418. In some embodiments, the arterial catheter assembly 412 may include an arterial catheter 420 extending from the distal end 416 of the catheter adapter 414. In some embodiments, the arterial catheter 420 may be shorter and/or more rigid than an intravenous catheter, such as a peripheral intravenous catheter.

In some embodiments, the arterial catheter system 410 may include a needle assembly 422, which may include a needle hub 424 and an introducer needle 426. In some embodiments, the arterial catheter system 410 may include a tube 428 coupled to the arterial catheter assembly 412 and having a distal end 430 and a proximal end 432. In some embodiments, the arterial catheter system 410 may include a fluid pathway 434 extending through at least the arterial catheter 420, catheter adapter 414, and the tube 428.

In some embodiments, a geometric factor G_{f} of a portion 436 of the fluid pathway 434 within the tube 428 may equal L/D⁴, wherein L is the length of the tube 428 and D is the inner diameter of the portion of the fluid pathway 434 within the tube 428. In these embodiments, the portion 436 of the fluid pathway 434 may be cylindrical along an entirety of the length L, and the inner diameter D may be constant along the length L. The length L corresponds to an entire length of the tube 428, according to some embodiments. In some embodiments, the geometric factor G_{f} of the portion 436 of the fluid pathway 434 within the tube 428 may be defined such that a fluidic resistance

$${R}_{f} = \frac{128 μ}{π} {G}_{f} ,$$

where

$${G}_{f} = \frac{L}{{D}^{4}} .$$

In some embodiments, the geometric factor G_{f} of the portion 436 of the fluid pathway 434 may be selected to reduce a maximum shear stress on arterial blood drawn from an artery and thereby reduce a risk of hemolysis of the arterial blood.

In some embodiments, the fluidic resistance may be a first fluidic resistance. In some embodiments, the first fluidic resistance of the portion 436 of the fluid pathway 434 within the tube 428 may be greater than a second fluidic resistance within the fluid pathway 434 distal to the portion 436 of the fluid pathway 434. For example, the first fluidic resistance may be greater than a particular fluidic resistance within a lumen 438 of the catheter adapter 414, where arterial blood may travel prior to reaching the tube 428 and adapter 440, which may be coupled to a blood collection device for blood collection.

As stated previously, a blood cell experiences shear stress as it flows in a fluid pathway. The maximum shear stress is along the wall of the fluid pathway, or wall shear stress. Wall shear stress on blood cells is considered a major source of mechanical damage to blood cells. For a cylindrical fluid path, the wall shear stress is typically expressed as:

$$τ = \frac{1}{2} \cdot \frac{Δ p}{L} \cdot ( kr )$$

in which ΔP is the pressure drop along a path with a length of L and an interior radius of r. k is shrinkage index.

To fill a certain volume of collection tube, V, with a flow rate of Q, the time needed can be simply assessed by:

$$t = \frac{v}{ℚ} = 8 μv \cdot \frac{1}{π {r}^{4}} / ( \frac{Δ p}{L} )$$

in which μ is the dynamic viscosity of the fluid. Hemolysis is typically associated with both the wall shear stress and the time a blood cell is exposed to wall shear stress. From literature, it has been widely considered that hemolysis index can be approached as a function of:

$$HI ( \% ) = A * {t}^{α} * {τ}^{β}$$

in which A, α, and β are coefficients.

In principle, the hemolysis index is related to pressure gradient and cross-sectional characteristic dimension:

$$HI ( \% ) ∝ {( \frac{Δ P}{l} )}^{β - α} \cdot {( \frac{1}{r} )}^{4 α - β}$$

Fluid flow in a particular tube with a cylindrical fluid pathway therethrough can be analyzed using Poiseuille's equation:

$$Q = \frac{π {D}^{4} Δ P}{128 μ L} = \frac{Δ P}{{R}_{f}}$$

where ΔP is a change in pressure gradient across the length of the tube, D and L are the inner diameter and length, respectively, of the cylindrical fluid pathway through the particular tube, μ is the viscosity of a fluid, and

$${R}_{f} = \frac{128 μ L}{π {D}^{4}}$$

is the fluidic resistance. The particular tube may include or correspond to the tube 428. Since μ is the viscosity of the fluid and not part of the tube geometry, the geometric factor G_{f} is defined such that R_{f} (the fluidic resistance) is

$${R}_{f} = \frac{128 μ}{π} {G}_{f} ,$$

where

$${G}_{f} = \frac{L}{{D}^{4}} .$$

In some embodiments, the tube 428 may have multiple sections with lengths (L1, L2, L3) and inner diameters of (D1, D2, D3), the geometric factor is then:

$${G}_{f} = \frac{L 1}{D {1}^{4}} + \frac{L 2}{D {2}^{4}} + \frac{L 3}{D {3}^{4}}$$

In some embodiments, the tube 428 may have an inner diameter that changes over the length of the tube, the geometric factor is then:

$${G}_{f} = {\int }_{0}^{L} \frac{dl}{{D ( l )}^{4}}$$

In some embodiments, the tube 428 may have a cross section that is not circular. In this case, the geometric factor can be determined by measuring the flow rate (Q) at given pressure (ΔP) with known viscosity (μ) fluid:

$${G}_{f} = \frac{π Δ P}{128 μ Q}$$

In some embodiments, the first fluidic resistance, which may be lower than the second fluidic resistance, may facilitate a decreased flow rate of arterial blood within the portion 436 of the fluid pathway 434 within the tube 428 such that the maximum shear stress is reduced within the portion 436 of the fluid pathway 434 and there is a decreased risk of hemolysis of the arterial blood that will be collected. In these embodiments, the length L and the inner diameter D of the tube 428, which may determine the geometric factor G_{f} of the portion 436 of the fluid pathway 434, may be selected to increase the first fluidic resistance and decrease the flow rate within the portion 436 of the fluid pathway such that the risk of hemolysis is decreased but the flow rate is still adequate for blood collection.

In some embodiments, the arterial catheter 420 may be a 20 G arterial catheter. In these embodiments, the length L and the inner diameter D may be selected such that the geometric factor G_{f} of the portion 436 of the fluid pathway 434 within the tube 428 is 3.33E+06 (1/in³) or higher. In these embodiments, the length L and the inner diameter D may be selected such that the geometric factor G_{f} of the portion 436 of the fluid pathway 434 within the tube 428 is 3.41E+06 (1/in³) or higher. In some embodiments, the length L and the inner diameter D may be selected such that the geometric factor G_{f} of the portion 436 of the fluid pathway 434 within the tube 428 is 3.33E+06 (1/in³) or higher.

In some embodiments, the arterial catheter 20 may be a 18 G arterial catheter. In these embodiments, the length L and the inner diameter D may be selected such that the geometric factor G_{f} of the portion of the fluid pathway within the tube 428 is 2.88E+06 (1/in³) or higher.

In some embodiments, the arterial catheter 20 may be a 22 G arterial catheter. In these embodiments, the length L and the inner diameter D may be selected such that the geometric factor G_{f} of the portion of the fluid pathway within the tube 428 is 1.05E+07 (1/in³) or higher.

In some embodiments, the arterial catheter 20 may be a 24 G arterial catheter. In these embodiments, the length L and the inner diameter D may be selected such that the geometric factor G_{f} of the portion of the fluid pathway within the tube 428 is 3.20E+07 (1/in³) or higher.

In some embodiments, the tube 428 may include no more than one lumen 442 extending therethrough. In these embodiments, a single lumen may be sufficient for the tube 428 in the arterial catheter system 410 since arterial catheters are rarely used for infusion where an extension tube with higher fluidic resistance might reduce the infusion rate significantly. In some embodiments, the catheter adapter 414 may include a side port 444 disposed between the distal end 416 of the catheter adapter 414 and the proximal end 418 of the catheter adapter 414. In some embodiments, an extension tube 454 may include a distal end coupled to or integrated with the side port 444. In some embodiments, the extension tube 454 may include a proximal end coupled to or integrated with an adapter 456, which may a Y-adapter or another suitable adapter. In some embodiments, the adapter 456 may include at least two ports, one for blood collection and the other for pressure monitoring. In some embodiments, a needleless access connector 457 may be coupled to one or more ports of the adapter 456.

In some embodiments, a compact connector 458 may include the tube 428 therein. In some embodiments, the tube 428 may include a spiral or coil shape, which may facilitate a compact device for easy use by a clinician. In some embodiments, the tube 428 may be flexible, rigid, or semi-rigid. In some embodiments, the tube 428 may be constructed of plastic or another suitable material. In some embodiments, a proximal end 460 of the compact connector 458 may include a female luer or another suitable adapter for coupling to a blood collection device. In some embodiments, the proximal end 460 may include a septum 461 configured to compress in response to coupling the proximal end 460 with the blood collection device. In some embodiments, in response to compressing of the septum 461, the blood collection device may be in fluid communication with the fluid pathway 434. In some embodiments, a distal end 462 of the compact connector 458 may be coupled to or integrated with the adapter 456.

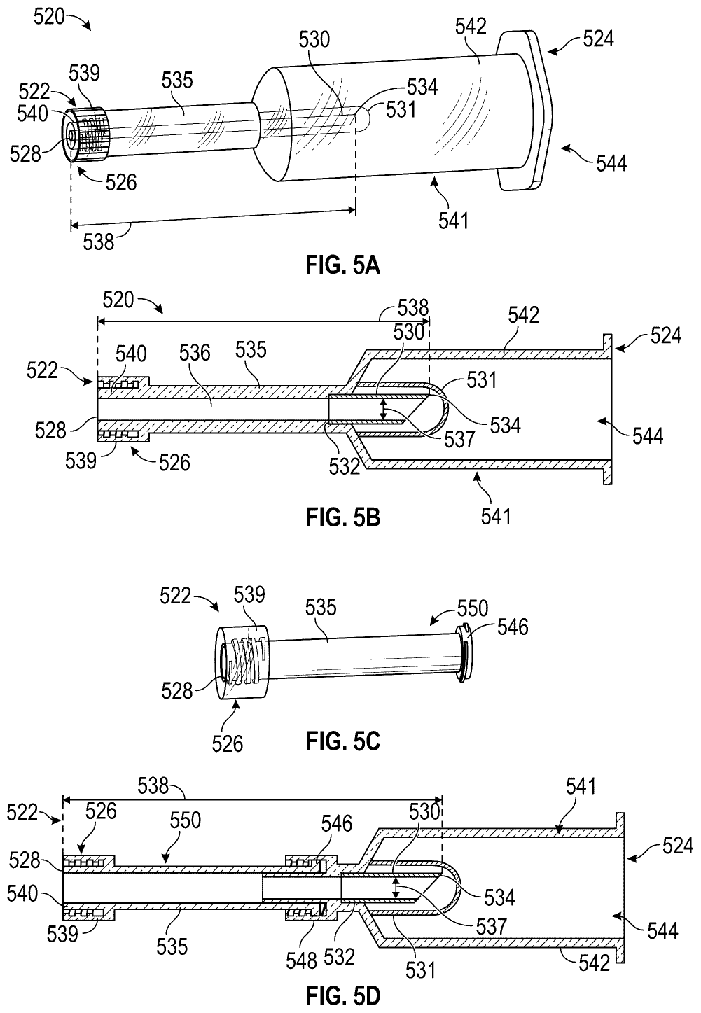

Referring now to FIGS. 5A-5B, a hemoshield device 520 is illustrated, according to some embodiments. In some embodiments, the hemoshield device 520 may include a distal end 522 and a proximal end 524. In some embodiments, the distal end 522 of the hemoshield device 520 may include a male luer adapter 526, which may include a distal opening 528. In some embodiments, the hemoshield device 520 may include a cannula 530 in fluid communication with the male luer adapter 526. In some embodiments, the cannula 530 may include a distal end 532 and a sharp proximal tip 534. In some embodiments, the cannula 530 may be constructed of metal or another suitable material configured to punctuate a seal of a blood collection container, such as a blood collection tube. In some embodiments, an elastomeric sheath 531, and in response to insertion of the blood collection container being inserted into the hemoshield device 520, the sharp proximal tip 534 may puncture the elastomeric sheath 531, which may be compressed in a distal direction by the blood collection container.

In some embodiments, the hemoshield device 520 may include an elongated neck 535 disposed between the male luer adapter 526 and the sharp proximal tip 534. In some embodiments, a fluid pathway of an arterial catheter system may include a portion 536 within the hemoshield device 520, the portion 536 extending from the distal opening 528 through the sharp proximal tip 534. In some embodiments, a diameter 537 of the portion 536 of the fluid pathway is constant. In some embodiments, an entire length 538 of the portion 536 of the fluid pathway is represented by L, and the diameter 537 of the portion 536 of the fluid pathway is represented by D.

In some embodiments, the male luer adapter 526 of the hemoshield device 520 may include a collar 539, which may extend around a protrusion 540 of the male luer adapter 526. In some embodiments, the distal opening 528 may be disposed within a distal-most portion of the protrusion 540. In some embodiments, an inner surface of the collar 539 may be threaded to form a luer-lock fit with a corresponding female luer adapter. In other embodiments, the inner surface of the collar 539 may be smooth to form a slip-fit with a corresponding female luer adapter. In some embodiments, the portion 536 of the fluid pathway may be formed entirely by the cannula 530, which may extend through the collar 539 and form the protrusion 540 and the distal opening 528.

In some embodiments, an outer diameter of the collar 539 may be greater than an outer diameter of the elongated neck 535. In some embodiments, the hemoshield device 520 may include a holder 541 configured to receive a blood collection container, such as a blood collection tube. In some embodiments, the holder 541 may include a cylindrical body 542. In some embodiments, the sharp proximal tip 534 may be disposed in a center of the cylindrical body 542 to facilitate puncture of a seal of the blood collection container in response to insertion of the blood collection container within a proximal opening 544 of the cylindrical body 542.

In some embodiments, an outer diameter of the cylindrical body 542 may be greater than the outer diameter of the elongated neck 535. In some embodiments, the elongated neck 535 may be disposed between the holder 541 and the collar 539. In some embodiments, the distal end 532 of the cannula 530 may be integrated and secured within the elongated neck 535. In some embodiments, the elastomeric sheath 531 may be coupled to an inner surface of the holder 541.

Referring now to FIGS. 5C-5D, in some embodiments, the hemoshield device 520 may include a female luer adapter 546 disposed at a proximal end of the elongated neck 535. In some embodiments, the distal end of the holder 541 may include a male luer adapter 548. In some embodiments, the female luer adapter 546 may be coupled to the male luer adapter 548. Thus, in some embodiments, the hemoshield device 520 may include an extension 550, which a user may couple to the holder 541 to provide the elongated neck 535 and an increased L, which may decrease the risk of hemolysis. In these and other embodiments, D may correspond to an inner diameter of the cannula 530. In some embodiments, an inner diameter of the elongated neck 535 may be equal to D along all or a portion of the elongated neck 535. In some embodiments, the male luer adapter 548 may be similar or identical to the male luer adapter 526 in terms of one or more features and/or operation.

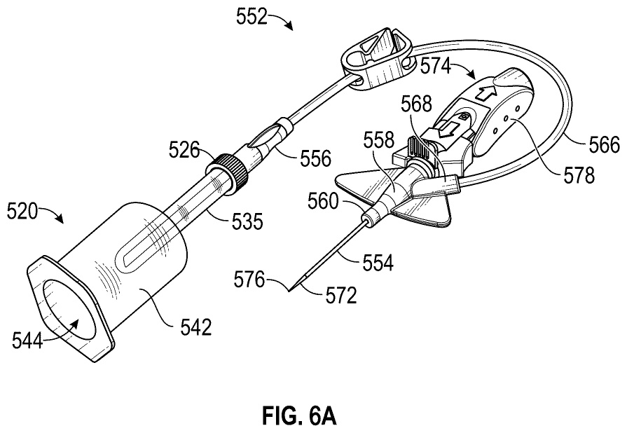

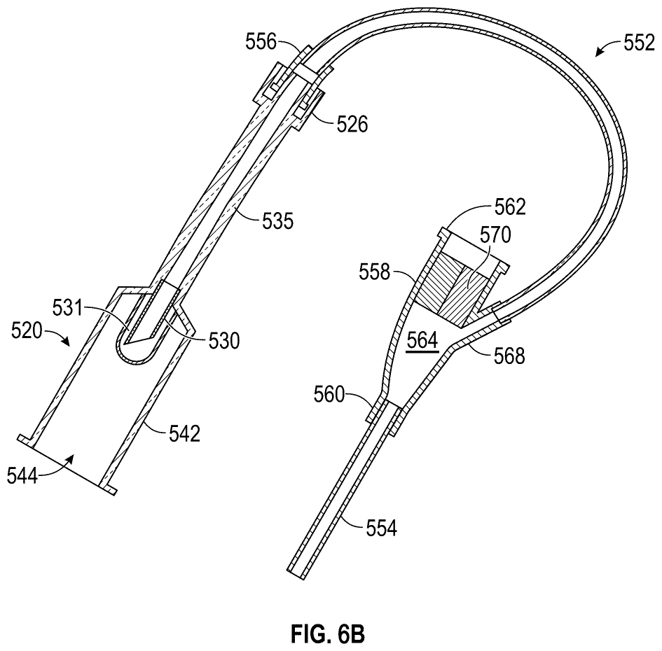

Referring now to FIGS. 6A-6B, an arterial catheter system 552 is illustrated, according to some embodiments. In some embodiments, the arterial catheter system 552 may be similar or identical to the arterial catheter system of one or more of FIGS. 1C, 3A, 3B, 4A, and 4B in terms of one or more included features and/or operation. In some embodiments, the arterial catheter system 552 may include an arterial catheter 554, and a female luer adapter 556 coupled to the arterial catheter 554. In some embodiments, the arterial catheter system 552 may include the hemoshield device 520, which may reduce the risk of hemolysis.

In some embodiments, the arterial catheter system 552 may include a catheter adapter 558, which may include a distal end 560, a proximal end 562, and a lumen 564 extending through the distal end 560 of the catheter adapter 558 and the proximal end 562 of the catheter adapter 558. In some embodiments, the arterial catheter 554 may extend distally from the distal end 560 of the catheter adapter 558.

In some embodiments, the male luer adapter 526 of the hemoshield device 520 may be coupled to the female luer adapter 556. In some embodiments, the arterial catheter system 552 and/or a location of the female luer adapter 556 may vary. In some embodiments, the arterial catheter system 552 may include an extension tube 66, which may include a distal end integrated with a side port 568 of the catheter adapter 558 and a proximal end integrated with the female luer adapter 556. In some embodiments, the side port 568 may be disposed in between the distal end 560 and the proximal end 562 of the catheter adapter 558 and in fluid communication with the lumen 564. In some embodiments, the proximal end 562 of the catheter adapter 558 may include the female luer adapter 556, and the hemoshield device 520 may be coupled to the proximal end 562 of the catheter adapter 558.

In some embodiments, a septum 570 may be disposed within the lumen 564 of the catheter adapter 558. In some embodiments, an introducer needle 572 of a needle assembly 574 may extend through the septum 570 and the arterial catheter 554 when the arterial catheter system 552 is inserted into the vasculature of the patient. In some embodiments, the needle assembly 574 may be removed from the arterial catheter system 552 in response to the arterial catheter 554 being inserted into the vasculature. In some embodiments, the introducer needle 572 may include a sharp distal tip 76 and may extend from a needle hub 578 of the needle assembly 574, which may be coupled to the proximal end 562 of the catheter adapter 558.

Typically, a maximum shear stress during blood collection through an arterial catheter 554 is much higher than a maximum shear stress during blood collection using another type of catheter, i.e. a venous catheter. Fluid flow in a particular cannula with a cylindrical fluid pathway can be analyzed using Poiseuille's equation:

$$Q = \frac{π {D}^{4} Δ P}{128 μ L} = \frac{Δ P}{{R}_{f}}$$

where ΔP is a change in pressure gradient across the length of the fluid pathway, D and L are the inner diameter and length, respectively, of the cylindrical fluid pathway through the particular cannula, μ is the viscosity of a fluid, and

$${R}_{f} = \frac{128 μ L}{π {D}^{4}}$$

is the fluid resistance. The particular cannula may include or correspond to the cannula 530, and the cylindrical fluid pathway may include or correspond to the portion 536. Since μ is the viscosity of the fluid and not part of the extension tube geometry, a geometric factor G_{f} is defined such that R_{f} (the fluid resistance) is

$${R}_{f} = \frac{128 μ}{π} {G}_{f} ,$$

where

$${G}_{f} = \frac{L}{{D}^{4}} .$$

In some embodiments, the portion 536 of the fluid pathway may have multiple sections with lengths (L1, L2, L3) and inner diameters of (D1, D2, D3), the geometric factor is then:

$${G}_{f} = \frac{L 1}{D {1}^{4}} + \frac{L 2}{D {2}^{4}} + \frac{L 3}{D {3}^{4}}$$

In some embodiments, the portion 536 of the fluid pathway may have an inner diameter that changes over the length of the fluid pathway, the geometric factor is then:

$${G}_{f} = {\int }_{0}^{L} \frac{dl}{{D ( l )}^{4}}$$

In some embodiments, the portion 536 of the fluid pathway may have a cross section that is not circular. In this case, the geometric factor can be determined by measuring the flow rate (Q) at given pressure (ΔP) with known viscosity (μ) fluid:

$${G}_{f} = \frac{π Δ P}{128 μ Q}$$

In some embodiments, the diameter of the portion 536 of the fluid pathway may be greater than a minimum inner diameter of the arterial catheter 554. In some embodiments, D⁴/L is equal to or less than a predetermined value, which may be based at least in part on the gauge of the arterial catheter 554. In some embodiments, the predetermined value may correspond to a value at which the risk of hemolysis is determined to below.

In some embodiments, the fluidic resistance within the portion 536 of the fluid pathway may facilitate a decreased flow rate of arterial blood within the portion 536 of the fluid pathway 534 within the cannula 530 such that the maximum shear stress is reduced and there is a decreased risk of hemolysis of the arterial blood that will be collected. In these embodiments, the length L and the inner diameter D of the cannula 530, which may determine the geometric factor G_{f} of the portion 536 of the fluid pathway, may be selected to increase the fluidic resistance and decrease the flow rate within the portion 536 of the fluid pathway such that the risk of hemolysis is decreased but the flow rate is still adequate for blood collection.

In some embodiments, a fluidic resistance within the portion 536 of the fluid pathway may be a first fluidic resistance. In some embodiments, the first fluidic resistance of the portion of the fluid pathway within the extension tube may be greater than a second fluidic resistance within the fluid pathway distal to the portion 536 of the fluid pathway. For example, the first fluidic resistance may be greater than a particular fluidic resistance within a lumen of the catheter adapter 564, where arterial blood may travel prior to reaching the extension tube 32 and the arterial hemoshield device 10 through which blood may be collected.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

We claim:

1. An arterial catheter system, comprising:
a catheter assembly, comprising:
a catheter adapter, comprising a distal end and a proximal end;
an arterial catheter extending from the distal end of the catheter adapter;
a needle assembly, comprising:
a needle hub;
an introducer needle;
an arterial hemoshield device coupled to the catheter assembly; and
a fluid pathway within the arterial catheter, the catheter adapter, and the arterial hemoshield device;
wherein a first fluidic resistance within a portion of the fluid pathway within the arterial hemoshield device is greater than a second fluidic resistance within the fluid pathway distal to the portion of the fluid pathway.

2. The arterial catheter system of claim 1, wherein the arterial hemoshield device comprises:
a distal end, comprising a luer adapter;
a proximal end, comprising a blood collection device; and
an extension tube extending between the distal end and the proximal end, wherein the portion of the fluid pathway is disposed within the extension tube.

3. The arterial catheter system of claim 2, wherein the extension tube comprises a distal end and a proximal end, wherein the distal end of the extension tube is integrated with the luer adapter, wherein the proximal end of the extension tube is integrated with the blood collection device.

4. The arterial catheter system of claim 2, wherein the luer adapter is a first luer adapter, wherein the blood collection device comprises a second luer adapter, further comprising a third luer adapter coupled to the second luer adapter, wherein the extension tube comprises a distal end and a proximal end, wherein the distal end of the extension tube is integrated with the first luer adapter, wherein the proximal end of the extension tube is integrated with the third luer adapter.

5. The arterial catheter system of claim 2, wherein a geometric factor G_{f} of the portion of the fluid pathway is different than a geometric factor G_{f} of another portion of the fluid pathway.

6. The arterial catheter system of claim 2, wherein the extension tube has no more than one lumen extending therethrough.

7. The arterial catheter system of claim 1, wherein the arterial hemoshield device comprises a compact connector, wherein the compact connector comprises a spiral tube, wherein the portion of the fluid pathway is disposed within the spiral tube.

8. The arterial catheter system of claim 7, wherein the catheter adapter further comprises a side port disposed between the distal end of the catheter adapter and the proximal end of the catheter adapter, wherein the catheter assembly further comprises another extension tube extending from the side port, wherein a distal end of the another extension tube is integrated with the side port, wherein a proximal end of the other extension tube is integrated with a Y-adapter, wherein the compact connector is coupled to the Y-adapter.

9. The arterial catheter system of claim 7, wherein a geometric factor G_{f} of the portion of the fluid pathway is different than a geometric factor G_{f} of another portion of the fluid pathway.

10. The arterial catheter system of claim 7, wherein the spiral tube has no more than one lumen extending therethrough.

11. The arterial catheter system of claim 1, wherein the arterial hemoshield device comprises,
a female luer adapter coupled to catheter assembly;
a blood collection device, comprising:
a distal end, comprising a male luer adapter coupled to the female luer adapter, the male luer adapter comprising a distal opening;
a cannula in fluid communication with the male luer adapter, wherein the cannula comprises a distal end and a sharp proximal tip; and
an elongated neck disposed between the male luer adapter and the sharp proximal tip,
wherein the portion of the fluid pathway extends from the distal opening through the sharp proximal tip.

12. The arterial catheter system of claim 11, wherein a geometric factor G_{f} of the portion of the fluid pathway is different than a geometric factor G_{f} of another portion of the fluid pathway.

13. A method of blood collection, comprising:
inserting an arterial catheter of an arterial catheter system into an artery of a patient, wherein the arterial catheter system comprises:
a catheter assembly, comprising:
a catheter adapter, comprising a distal end and a proximal end;
an arterial catheter extending from the distal end of the catheter adapter;
a needle assembly, comprising:
a needle hub;
an introducer needle;
an arterial hemoshield device coupled to the catheter assembly;
a fluid pathway within the arterial catheter, catheter adapter, and the arterial hemoshield device, wherein a first fluidic resistance within a portion of the fluid pathway within the arterial hemoshield device is greater than a second fluidic resistance within the fluid pathway distal to the portion of the fluid pathway; and
collecting arterial blood in a blood collection device coupled to the catheter assembly, whereby the arterial blood flows through the portion of the fluid pathway and into the blood collection device.

14. The method of claim 13, wherein the arterial hemoshield device comprises:
a distal end, comprising a luer adapter;
a proximal end, comprising a blood collection device; and
an extension tube extending between the distal end and the proximal end, wherein the portion of the fluid pathway is disposed within the extension tube.

15. The method of claim 13, wherein the arterial hemoshield device comprises a compact connector, wherein the compact connector comprises a spiral tube, wherein the portion of the fluid pathway is disposed within the spiral tube.

16. The method of claim 13, wherein the hemoshield device comprises:
a female luer adapter coupled to the arterial catheter;
a blood collection device, comprising:
a distal end, comprising a male luer adapter coupled to the female luer adapter, the male luer adapter comprising a distal opening;
a cannula in fluid communication with the male luer adapter, wherein the cannula comprises a distal end and a sharp proximal tip; and
an elongated neck disposed between the male luer adapter and the sharp proximal tip,
wherein the portion of the fluid pathway extends from the distal opening through the sharp proximal tip.

17. A method of manufacturing an arterial catheter system having a fluid pathway therein, the method comprising:
coupling a catheter assembly to a needle assembly, wherein the catheter assembly comprises a catheter hub and an arterial catheter extending distally from the catheter hub;
coupling an arterial hemoshield device to the catheter assembly such that the arterial hemoshield device is in fluid communication with the catheter assembly and the fluid pathway is within the arterial catheter, the catheter hub, and the arterial hemoshield device; and
selecting a length L of a portion of the fluid pathway within the arterial hemoshield device and an inner diameter D of the portion of the fluid pathway within the arterial hemoshield device such that a first fluidic resistance within the portion of the fluid pathway is greater than a second fluidic resistance within the fluid pathway distal to the portion of the fluid pathway.
